SEQUENCE LISTING (2 sequences)

SEQ ID NO: 1 - DNA, 10 bp, artificial sequence
  primer for the random amplification of chromosomal and plasmidic bacteria DNA
agcagcgtgg

SEQ ID NO: 2 - DNA, 15 bp, artificial sequence
  primer for the random amplification of chromosomal and plasmidic bacteria DNA
gagggtggcggttct

(19) United States
(12) Patent Application Publication
(10) Pub. No.: US 2013/0231291 A1
(43) Pub. Date: Sep. 5, 2013

(54) FERMENTED SOYA BASED MIXTURE COMPRISING ISOFLAVONES- AGLICONES, EQUOL AND LUNASIL, PROCESS FOR THE PREPARATION AND USES THEREOF IN FOOD, MEDICAL AND COSMETIC FIELDS

(71) Applicants: Giammaria Giuliani, Milano (IT); Anna Benedusi, Milano (IT); Marco Gobbetti, Milano (IT); Raffaella Di Cagno, Milano (IT); Sergio Baroni, Milano (IT); Carlo Giuseppe Rizzello, Bari (IT)

(72) Inventors: Giammaria Giuliani, Milano (IT); Anna Benedusi, Milano (IT); Marco Gobbetti, Milano (IT); Raffaella Di Cagno, Milano (IT); Sergio Baroni, Milano (IT); Carlo Giuseppe Rizzello, Bari (IT)

(21) Appl. No.: 13/806,746

(22) PCT Filed: Jul. 11, 2011
(86) PCT No.: PCT/IT2011/000240

§ 371 Date: May 14, 2013

(30) Foreign Application Priority Data
Jul. 12, 2010 (IT) RM2010A000378

(51) Int. Cl. A23L 1/20; A61K 38/16; A61K 8/64; C12P 21/00

(52) U.S. Cl. CPC A23L 1/2008; C12P 21/00; A61K 38/168; A61K 8/645
USPC 514/18.8; 435/71.2; 514/1.1; 514/18.6; 514/20.7; 435/252.9; 426/634

(57) ABSTRACT

The present invention concerns a mixture comprising isoflavones-aglicones, such as daidzein, genistein and glycitein, in addition to equol and lunasin, said mixture being based on soya fermented using lactic acid bacteria isolated from food matrices and use of said mixture for protection of skin and similar and of human intestinal cells with particular reference to prevention of inflammatory state and protection of barrier functions and hair loss treatment.

The present invention concerns a fermented soya based mixture comprising isoflavones-aglicones, equol and lunasil, the process for the preparation and uses thereof in food, medical and cosmetic fields. In particular, the present invention concerns a mixture comprising isoflavones-aglicones, such as daidzein, genistein and glycitein, in addition to equol and lunasin, said mixture being based on soya fermented using lactic acid bacteria isolated from food matrices and use of said mixture for protection of skin and adnexa and of human intestinal cells with particular reference to prevention of inflammatory state and protection of barrier functions and hair loss treatment.

Isoflavones are diphenolic compounds naturally occurring in various plants and particularly soya (Tsangalis et al., 2002. Enzymatic transformation of isoflavone phytoestrogens in soymilk by β-glucosidase producing bifidobacteria. Food Res. Int. Sci. 67:3104-3113). Soya derived isoflavones and soya based food products belong to 4 classes of chemical compounds: aglicones, malonyl-, acetyl- and β-glucoside-conjugates (Tsangalis et al., 2002. Enzymatic transformation of isoflavone phytoestrogens in soymilk by β-glucosidase producing bifidobacteria. Food Res. Int. Sci. 67:3104-3113). More than 90% of soya isoflavone total concentration occurs as β-glucoside derivatives. Because of the remarkable hydrophobic character and high molecular mass, 3-glucoside derivatives are not absorbed by humans. Therefore in order to be bioavailable and thus metabolised said compounds must be hydrolysed to aglicones. Hydrolysis to aglicones such as daidzeindaidzein, genistein and glycitein occurs during the intestinal passage as result of activity of intestinal and bacterial β-glucosidase. In free form, such aglicones are structurally similar to estrogens and mimic the estradiol function in human body (Setchell and Cassidi, 1999. Dietary isoflavones: biological effects and relevance to human health. J. Nutr. 131:758-767). Generally, the consumption of isoflavones-aglicones is associated with the reduction of risk of hormonal pathologies (Kruzer 2000. Hormon effects of soy isoflavones: studies in premenopausal and postmenopausal women. J. Nutr. 130: 660-661), and, with lower incidence of osteoporosis, menopause and mortality from cardiovascular pathologies and cancer (Nagata et al. 1998. Decreased serum total cholesterol concentration is associated with high intake of soy products in Japanase men and women. J. Nutr. 128:209-213).

Equol is an estrogen not steroidal compound belonging to isoflavone class. The main source of equol for humans is soya derivatives, representing most abundant reserve for daidzein and aglicone daidzein, direct precursor thereof (Axelson et al., 1984. Soya a dietary source of the non-steroidal oestrogen equol in man and animals. J. Endocrinol. 102:49-56). Differently than other isoflavones-aglicones, equol is the only one having a core chiral nucleus resulting from the absence of double bond within heterocyclic ring (Setchell et al., 2002. The clinical importance of the metabolite equol a clue to the effectiveness of soy and its isoflavones. Am. Soc. Nutr. Sci. 125: 3577-3583). Generally, equol is absorbed easily through colon wall and is metabolically inert (Setchell et al., 2002. The clinical importance of the metabolite equol a clue to the effectiveness of soy and its isoflavones. Am. Soc. Nutr. Sci. 125: 3577-3583). Compared to daidzein precursor thereof equol shows an interesting set of properties: higher estrogenic activity (Muthyala et al., 2004. Equol, a natural estrogenic metabolita from soy isoflavones: convenient preparation and resolution of R- and S-equols and their differing binding and biological activity through estrogen receptors alpha and beta. Bioorg. Med. Chem. 12:1559-1567) anti-oxidant activity (Mitchell et al., 1998. Antioxidant efficacy of phytoestrogens in chemical and biological model systems. Arch. Biochem. Biophys. 360:142-148), and antiandrogenic activity (Lund et al., 2004. Equol is a novel anti-androgen that inhibits prostate growth and hormone feedback. Bio. Reprod. 70:1188-1195).

Soya isoflavone metabolism in man is widely documented (Axelson et al., 1984. Soya a dietary source of the non-steroidal oestrogen equol in man and animals. J. Endocrinol. 102:49-56; Bannwart et al., 1984. Identification of o-desmethylangolensin, a metabolita of daidzein and of matairesinol, one likely plant precursor of the animal lignan enterolactone in human urine. Finn. Chem. Lett. 5:120-125). The ability to metabolise glucoside isoflavones to aglicones and aglicones to equol during intestinal passage is limited only to 30-50% of the western countries population (Frankefeld, et al. 2005. High concordance of daidzein-metabilizing phenotypes in individuals measured 1 to 3 years apart. Brit. J. Nutr. 94:873-876). Two main strategies can be pursued in order to increase the bioavailability of soya derived isoflavones: aglicone and equol enrichment before the consumption or modulation of intestinal microbiota by ingestion of viable and vital bacteria suitable to synthesise in situ such compounds (Tsangalis et al., 2004. Development o fan isoflavone aglycone-enriched soymilk using soy germ, soy protein isolate and bifidobacteria. Food Res. Intern. 37:301-312). Various studies (Chun et al., 2007. Conversion of isoflavone glucoside to aglycones in soymilk by fermentation with lactic acid bacteria. J. Food Sci. 72:39-44; Donkor and Shah 2008. Production of β-glucosidase and hydrolysis of isoflavone phytoestrogens by Lactobacillus acidophilus, Bifidobacterium lactis and Lactobacillus casei in soymilk. J. Food Sci. 73:15-20; Pham and Shah 2007. Biotransformation of isoflavone glycosides by Bifidobacterium animalis in soymilk supplemented with skim milk powder. J. Food Sci. 72:316-324; Tsangalis et al., 2002; Tsangalis et al., 2004; Wei et al., 2007. Using Lactobacillus and Bifidobacterium to product the isoflavone algycones in fermented soymilk. Int. J. Food Microbiol. 117:120-124) have considered the use of bifidobacteria and lactic acid bacteria for the conversion of glucoside isoflavones to aglicones and/or equol during the fermentation of soya milk. However, some limitations are apparent in these studies: (i) a very limited number of bacterial biotype/species has been considered; (ii) bacteria used for fermentation processes are exclusively originated from human fecal material; (iii) a very limited number of substrates for fermentation, none of which involved the use of soya biological flour has been considered; (iv) preparations are based only on isoflavone/aglicones or equol, and in the case of equol production maximum concentration is 0,521 mg/100 ml (Tsangalis et al., 2002. Enzymatic transformation of isoflavone phytoestrogens in soymilk by β-glucosidase producing bifidobacteria. Food Res. Int. Sci. 67:3104-3113); (v) no study tested the biological effectiveness of preparations, in particular for skin protection; and (vi) no study formulated a preparation containing isoflavones-aglicones, equol and lunasin.

Lunasin is a bioactive peptide (43 aminoacid residues, molecular weight about. 5400 Da) identified for the first time in soya (Galvez et al., 2001. Chemopreventive property of a soybean peptide (Lunasin) that binds to deacetylated histones and inhibits acetylation. Cancer Res. 61:7473-7478) and successively found also in barley (Jeong et al. 2002. Barley lunasin suppresses ras-induced colony formation and inhibits core histone acetylation in mammalian cells. J. Agric. Food Chem. 50:5903-5908), wheat (Jeong et al. 2007. The cancer preventive peptide lunasin from wheat inhibits core histone acetylation. Cancer Lett. 255:42-48), and amaranth (Silva-Sanchez et al., 2008. Bioactive peptides in amaranth (Amaranthus hypochondriacus) seed. J. Agric. Food Chem. 56:1233-1240). The concentration of lunasin in soya can vary depending on the cultivar, culture pedoclimatic atmosphere and technological processes grains have been subjected to after the harvesting. Lunasin very high concentration has been found in Loda cultivar (about 11 mg/g), while in other soya varieties (for example. Imari) lunasin content does not exceed 5-6 mg/g (Wang et al. 2008. Analysis of soybean protein derived peptides and the effect of cultivar, environmental conditions, and processing of lunasin concentration in soybean and soy products. J. AOAC Intern. 91:936-944). Lunasin contains 9 aspartic acid residues at C-terminus of polypeptide chain. This composition favours an elevated affinity to hypo-acetylated chromatin regions, to which the peptide can bind thus inhibiting acetylation-deacetylation dynamics and, therefore, acting as tumour suppressor in carcinogenesis. It has been also reported that lunasin can exert a prevention activity against carcinogenesis phenomena, thanks to inhibition of cell proliferation induced by ras gene and to acetylation inhibition of H3 histone (Jeong et al., 2003. Characterization of lunasin isolated from soybean. J Agric Food Chem. 51: 7901-7906). From literature data it it is apparent that no study has considered up to now lunasin enrichment for soya derivatives by fermentation processes using lactic acid bacteria.

Based on above reported considerations, some elements appear to display a marked innovative character: (i) to employ soya based substrates, possibly of biological origin; (ii) to employ lactic acid bacteria isolated from food matrices and not of fecal origin; (iii) to optimize a biotechnological process suitable to favour the formulation of a preparation containing higher number of functional molecules such isoflavones-aglicones, equol and lunasin; (iv) to demonstrate, using in vitro and ex vivo assays, the effect of resulting preparation as to cutaneous and human intestinal cell protection, with particular reference to inhibition of inflammatory state and barrier function keeping.

The authors of the present invention now developed a new process suitable to provide a fermented soya based mixture enriched for isoflavones, aglicones, equol and lunasin. The mixture according to the invention is obtained by fermentation of soya using a particular mixture of lactic acid bacteria exclusively derived from food matrices and not of fecal origin. The mixture according to the invention, due to high content of isoflavones-aglicones, equol and lunasin, in particular lunasin, displayed particular effectiveness for cutaneous and human intestinal cell protection with particular reference to the prevention of inflammatory state and barrier function keeping.

The authors of the present invention have now considered that: (i) the selection of 103 isolated lactic acid bacteria, derived exclusively from food matrices, according to β-glucosidase activity on p-nitrophenyl-6-D-glucopyranoside (pNPG) allowed the selection of 4 lactic acid bacteria, namely L. plantarum DPPMA24W (deposited at DSMZ on 8 Jul. 2010 DSM number 23756) and DPPMASL33 (deposited at DSMZ on 8 Jul. 2010 DSM number 23755), L. fermentum DPPMA114 (deposited at DSMZ on 8 Jul. 2010 DSM number 23757) and L. rhamnosus DPPMAAZ1 (deposited at DSMZ on 8 Jul. 2010 DSM number 23758), to be used as mixed starter for fermentation of soya flour based substrates; (ii) the use of 14 different soya based substrates allowed to select as optimal the preparation based on biological soya flour for fermentation using mixed starter; (iii) the optimization of fermentation process of biological soya flour substrate allowed the formulation of a preparation comprising 1.45 mg/100 ml (57.0 μM) of daidzein, 3.9 mg/100 ml (140.3 μM) of genistein, 0.58 mg/100 ml (20.4 μM) of glycitein, 0.9 mg/100 ml (37.3 μM) of equol and 8.4 mg/100 ml of lunasin; (iv) the preparation based on above-mentioned functional compounds displays a protection effect on the cutaneous epidermis and positive effect on the inhibition of inflammatory state and barrier functions of intestinal cells.

Lactic acid bacteria according to the present invention belong to the Lactobacillus species and have been isolated from natural yeasts used for bread-making in Central and Southern Italy and from aged “pasta filata” cheeses of Pecorino type from Puglia region. Generally, the lactic acid bacteria isolated from such food matrices display metabolic and environmental adaptation characteristics not too much dissimilar than microorganisms colonizing gastrointestinal tract of humans and animals. L. plantarum DPPMA24W (deposited at DSMZ on 8 Jul. 2010 DSM number 23756) and L. plantarum DPPMASL33 (deposited at DSMZ on 8 Jul. 2010 DSM number 23755), L. fermentum DPPMA114 (deposited at DSMZ on 8 Jul. 2010 DSM number 23757) and L. rhamnosus DPPMAAZ1 (deposited at DSMZ on 8 Jul. 2010 DSM number 23758) have been selected and used.

A biotechnological protocol involving the fermentation by means said four lactic acid bacteria on various soya flour based substrates, preferably of biological origin for 48-96 h at 30-37° C. has been standardized and optimized. At the end of fermentation process, cells can be removed or not from culture broth by means of centrifugation and subjecting the supernatant to a dehydration process by drying or freeze-drying.

Below biotechnological protocol for fermentation of the biological soya based preparation is described.

Propagation of selected 4 lactic acid bacteria cultures at 30° C. for 24 h, washing, water suspension at a cellular density of 9.0 log ufc/ml and inoculum (1-4%) of soya milk (various soya flours, preferably biological soya)

↓
Culture at 30-37° C. for 48-96 h
↓
Cell removal by centrifugation
↓
Supernatant dehydration by drying or freeze-drying
↓
Formulation of the preparation for medical applications

As a result of fermentation of various soya milk preparations the synthesis of 3.9-57.0 μM of daidzein, 7.8-140.3 μM of genistein, 6.7-20.4 μM of glycitein, 7.6-37.3 μM of equol and about 8.4 mg/100 ml of lunasin has been obtained. Upper limits of above reported concentrations refer to fermentation of soya milk derived from biological soya flour. According to one of possible formulations, the application of fermented products from biological soya displayed to be suitable to: (i) protect epidermis enhancing barrier functions thereof; (ii) inhibit the inflammatory state of Caco-2/TC7 cells following the induction by γ-interpheron (IFN-γ) and lipopolysaccharide (LPS); (iii) stimulate barrier functions as demonstrated by Transepithelial Electric Resistance (TEER) test; and (iv) inhibit the synthesis of interleukin-8 (IL-8).

As demonstrated by complementary analysis using microbiological, chromatographic techniques and assays on in vitro and ex vivo cell cultures, the fermentation of soya biological flour by mixed starter consisting of lactic acid bacteria species isolated from food matrices and not used in previous studies, according to the present invention, allows: (i) the concomitant synthesis of aglicones, equol and lunasin, not found in previous studies and (ii) a protective effect against inflammatory state, enhancing the barrier function of epidermis and intestinal human cells.

It is therefore, a specific object of the present invention a process for the preparation of a fermented soya based mixture, comprising isoflavones-aglicones, equol and lunasin, by soya fermentation using a mixture of the following four lactic acid bacteria: Lactobacillus plantarum DSM 23755, Lactobacillus plantarum DSM 23756, Lactobacillus fermentum DSM 23757 and Lactobacillus rhamnosus DSM 23758. The mixture obtained according to the process of the invention is a mixture enriched for isoflavones-aglicones, lunasin, equol, that is it contains a greater percentage of these compounds in comparison to known mixtures obtained by processes using lactic acid bacteria different than those of the present invention.

The process according to the invention comprises or consists in the following steps: a) culture propagating said four Lactobacillus plantarum DSM 23755, Lactobacillus plantarum DSM 23756, Lactobacillus fermentum DSM 23757 and Lactobacillus rhamnosus DSM 23758 lactic acid bacteria;

b) inoculating soya based substrates with an aqueous suspension of said lactic acid bacteria; preferably the substrates are inoculated with aqueous suspension of lactic acid bacteria in an amount from 1 to 4% of total substrate volume, said aqueous suspension having a cell density about log 9.0 ufc/ml for each biotype;

c) incubating at 30-37° C., preferably 30° C., for 48-96 h, preferably 96 h.

Soya based substrates suitable to be used are, for example, soya flour, preferably biological soya flour, soya milk and other commercial formulations as reported according to present application.

The process according to the invention can further comprise the step d) of centrifugation of broth-culture in order to separate cells from lactic acid bacteria. In particular, the centrifugation of the broth-culture can be carried out at 10.000×g for 15 min at 4° C.

The process according to the present invention can further comprise a step e) of dehydration of supernatant obtained in step d) by drying or freeze-drying. According to an embodiment the formulation can contain viable, vital lactic acid bacteria omitting step d). The preparation of a composition can involve, at the end of the dehydration process, the formulation with addition of suitable excipients in order to obtain the preparation of forms suitable to the oral or topical use depending on circumstances.

It is a further object of the present invention a mixture, comprising isoflavones-aglicones, equol and lunasin, based on fermented soya obtainable according to the process as above defined without step d) of removal of lactic acid bacteria. Said mixture, therefore, contains above mentioned lactic acid bacteria according to the present invention. As above reported, the mixture according to the invention is a mixture enriched for isoflavones-aglicones, equol, and lunasin, that is, it contains an higher percentage of these compounds than known mixtures obtained by processes using lactic acid bacteria different than those of the present invention.

The present invention concerns, moreover, a pharmaceutical or cosmetic composition comprising or consisting of the mixture as above defined together with one or more pharmaceutically or cosmetically acceptable excipients and/or adjuvants.

According to a further embodiment, the mixture according to the invention can be used as a food integrator. For example the mixture could be used also for traditional foods, for example bake o pasta products.

Moreover, the mixture or the composition according to the invention can be used for the treatment of disorders or diseases of the skin or intestine. In particular, said mixture or composition can be used against modifications of skin barrier function, for example for prevention or treatment of sensitive skin, dried skin, psoriasis, atopic dermatitis, seborrheic dermatitis, dandruff, irritative dermatosis, eczema dermatosis, contact dermatosis, ulcers, acne, skin aging. Moreover, the mixture or composition according to the invention can be used in case of modification of intestinal barrier function, for example for the treatment or the prevention of the celiac disease, food intolerances, Crohn's disease.

The mixture or composition according to the invention can be used in cosmetic field, for example for treatment of hair loss or in medical field for the treatment of alopecia or telogen defluvium.

Particularly, the mixture or composition according to the invention can be administered by topical way, for example in form of creams, lotions, pastes, salves, gel, solutions, emulsions, suspensions or systemically, for example by oral way, for example as vial, chewable tablet, pill, sachet, etc.

Of course also the mixture obtained according to the process of invention comprising the step d) of removal of the lactic acid bacteria or a pharmaceutical or cosmetic composition containing the same can be advantageously used for above reported indications because it contains an higher percentage of isoflavones-aglicones, equol and lunasin, than known mixtures obtained by means of processes using lactic acid bacteria different than those of the present invention.

Moreover, a mixture of following four lactic acid bacteria, Lactobacillus plantarum DSM 23755, Lactobacillus plantarum DSM 23756, Lactobacillus fermentum DSM 23757 and Lactobacillus rhamnosus DSM 23758 is an object of the present invention. Finally Lactobacillus plantarum DSM 23755 or Lactobacillus plantarum DSM 23756 or Lactobacillus fermentum DSM 23757 or Lactobacillus rhamnosus DSM 23758 is an object of the present invention.

The present invention now will be described by an illustrative, but not limitative way, according to preferred embodiments thereof, with particular reference to enclosed drawings.

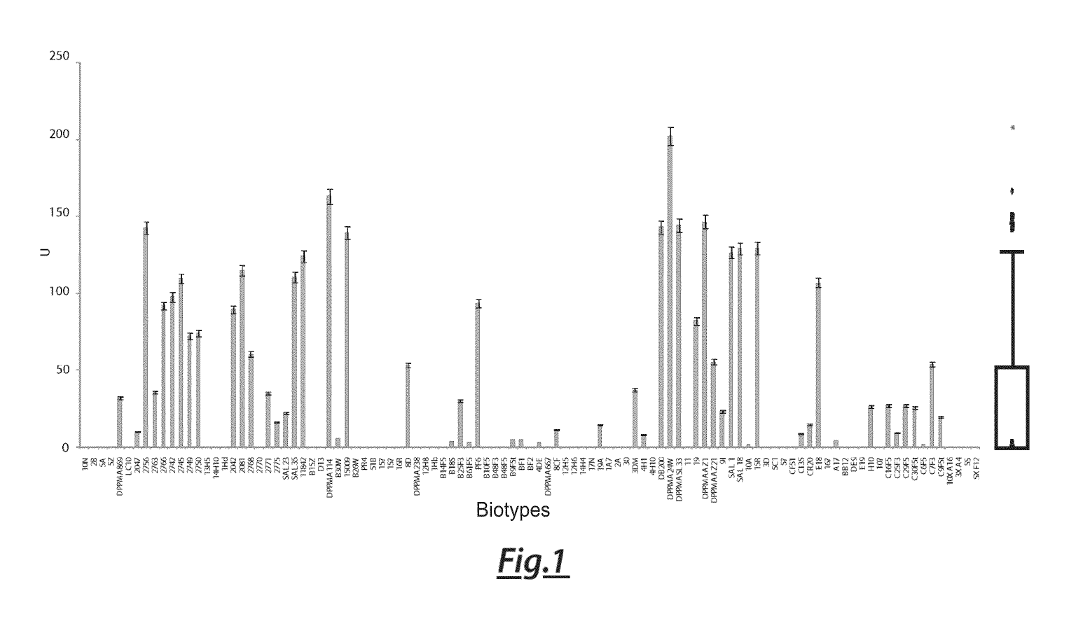

FIG. 1 shows β-glucosidase activity of 103 lactic acid bacteria biotypes belonging to various species on pNPG synthetic substrate. All the lactic acid bacteria used in the assay have been previously isolated, in absolutely innovative way, only from food matrices. Lactic acid bacteria biotypes are indicated by code, in order to identify the correspondence thereof to the species, please refer to protocol description in the text (Example 1). Data are the average of three triplicate experiments. Statistical elboration by box plot is reported

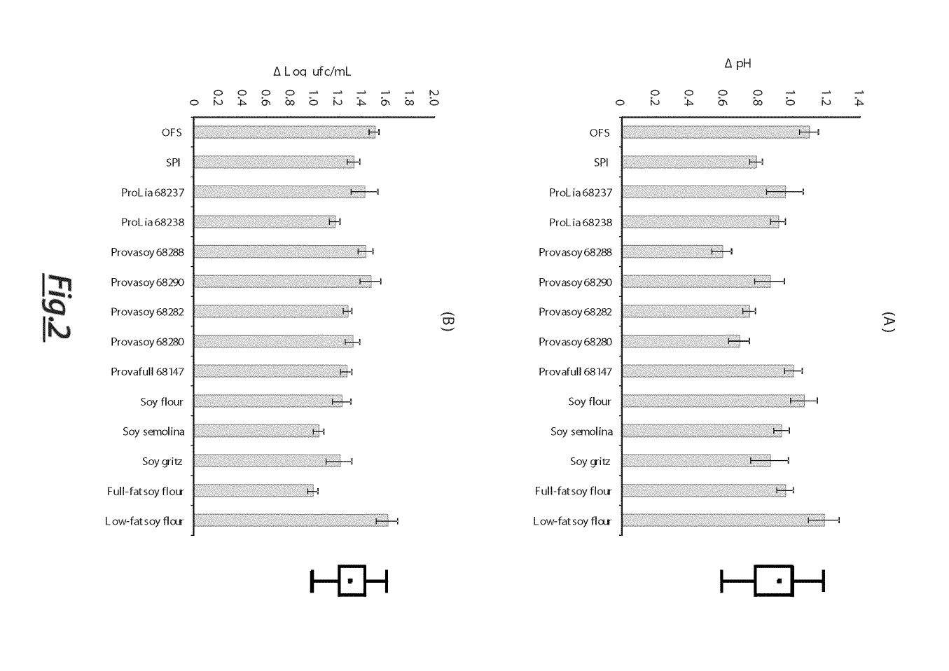

FIG. 2A shows the lactic acidification process carried out by mixed starter selected in the presence of soya milk from 14 different flours. FIG. 2B shows the cell density of lactic acid bacteria biotypes comprising the mixed starter selected in the presence of soya milk from 14 different flours. Data are the average of three triplicate experiments. Statistical elaboration by box plot is reported

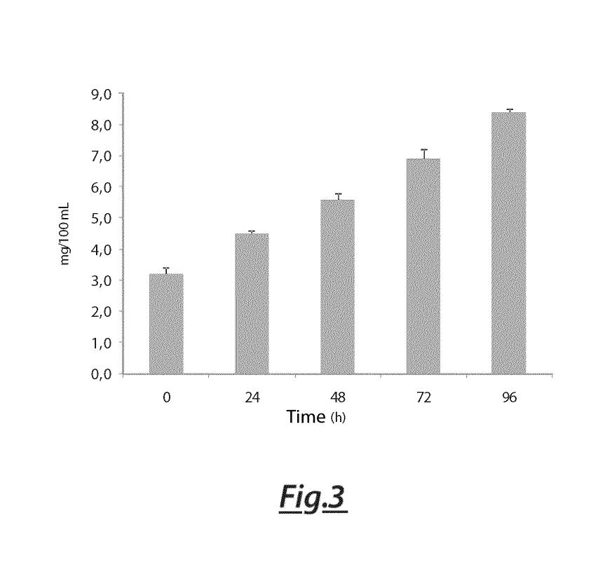

FIG. 3 shows the synthesis of lunasin (mg/100 ml) during the fermentation of soya milk obtained from biological soya flour (OFS) using selected mixed starter. Data are the average of three triplicate experiments.

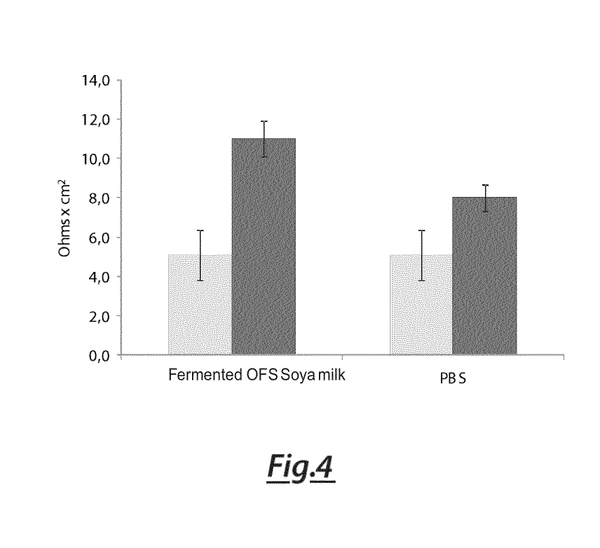

FIG. 4 shows Transepithelial Electric Resistance (TEER) (Ohms×cm²) of reconstituted epidermis (SkinEthic®) after exposure for 0 and 24 h to biological fermented soya milk (OFS) using the selected mixed starter and PBS buffer. Data are the average of three triplicate experiments.

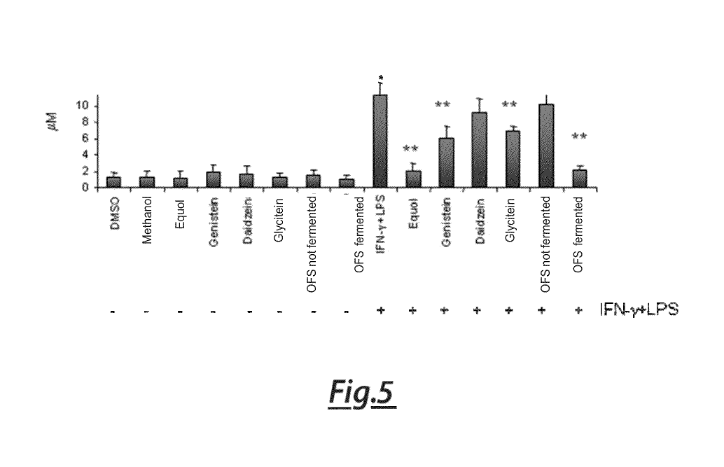

FIG. 5 shows nitric oxide release (μM) (NO) from Caco-2/TC7 cells. The cells have been pre-treated for 24 h with chemical compounds (10 μM) used as standards (equol, daidzein, genistein and glycitein) and soya milk, obtained from biological soya flour, not fermented or fermented using mixed selected starter, diluted at equol final concentration of 10 μM and sterile filtered. Successively, the cells have been stimulated with γ-interpheron (IFN-γ) (1000 U/ml) and lipopolysaccharide (LPS) (100 ng/ml) for 24 h. DMEM culture medium containing DMSO (1%, v/v) or methanol (0.5%, v/v) has been used as negative control. Data are the average of three triplicate experiments. Asterisk indicates meaningful differences (P<0.01) compared to negative control.

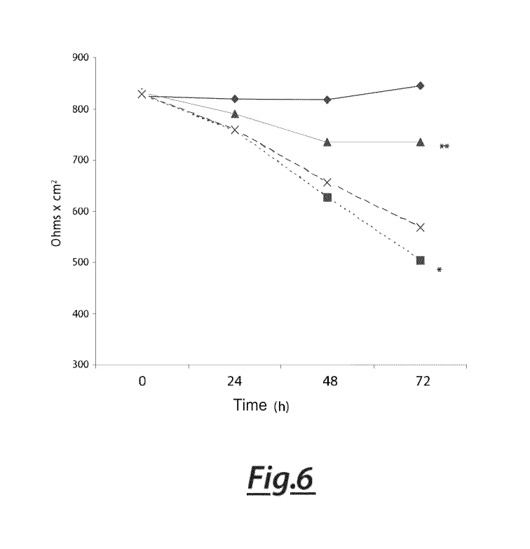

FIG. 6 shows the Transepithelial Electric Resistance (TEER) (Ohms×cm²) of Caco-2/TC7 cells after 24, 48 and 72 h of incubation. The incubation has been carried out in the presence of γ-interpheron (IFN-γ) (1000 U/ml (-▪-); IFN-γ and soya milk, obtained from biological soya flour, not fermented (-Δ-) or fermented with the selected mixed starter, diluted at equol final concentration of 10 μM and sterile filtered. (-x-). DMEM culture medium has been used as negative control (-♦-). Data are the average of three triplicate experiments. Asterisk indicates meaningful differences (P<0.01) compared to negative control.

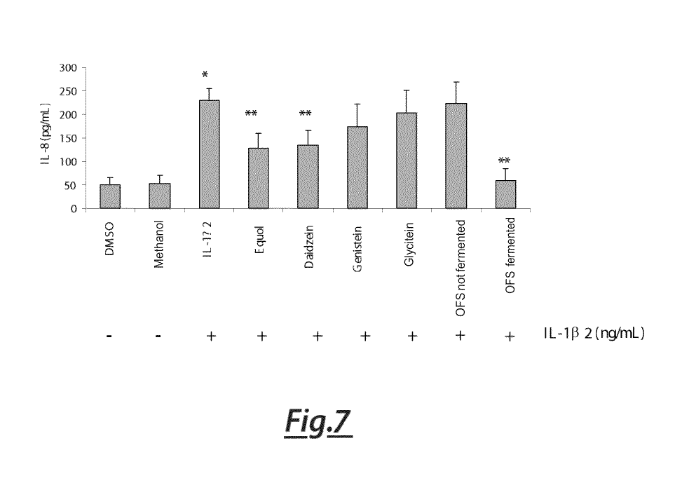

FIG. 7 shows the release (pg/ml) of interleukin-8 (IL-8) from Caco-2/TC7 cells stimulated for 24 h with interleukin-1β (IL-1β) (2 ng/ml) and successively treated (24 h) with chemical compounds (10 μM) used as standards (equol, daidzein, genistein and glycitein) and with soya milk, obtained from biological soya flour, not fermented or fermented with the selected mixed starter, diluted at equol final concentration of 10 μM and sterile filtered. DMEM culture medium containing DMSO (1%, v/v) or methanol (0.5%, v/v) has been used as negative control. Data are the average of three triplicate experiments. Asterisk indicates meaningful differences (P<0.01) compared to negative control.

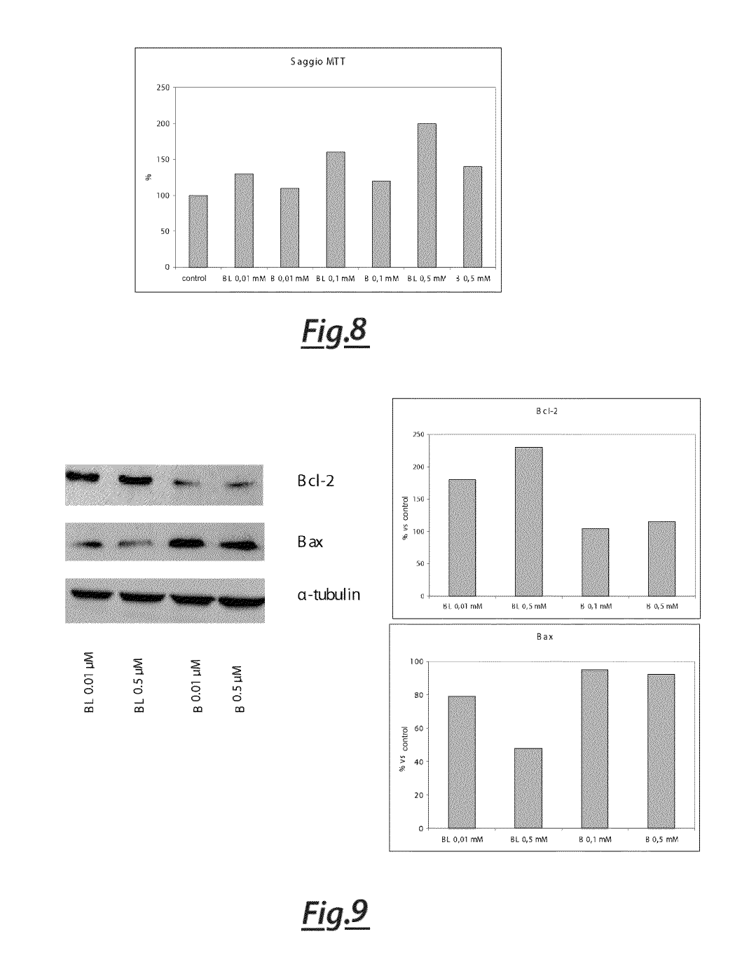

FIG. 8 shows the effect of biomass containing lunasin and without lunasin on the cell proliferation.

FIG. 9 shows the effect of the biomass containing lunasin and without lunasin on the protein expression of Bcl-2 and Bax.

### EXAMPLE 1

### β-Glucosidase Activity of 103 Biotypes of Lactic Acid Bacteria Isolated from Food Matrices

One hundred and three biotypes of lactic acid bacteria belonging to Lactobacillus alimentarius (10N, 2B, 5A), Lactobacillus brevis (5Z, DPPMA869), Lactobacillus casei (LC10), Lactobacillus casei subsp. casei (2047, 2756, 2763, 2766), Lactobacillus casei subsp. pseudoplantarum (2742, 2745, 2749, 2750), Lactobacillus curvatus (13H5, 14H10, 1Hd, 2042, 2081, 2768, 2770, 2771, 2775, SAL23, SAL35), Lactobacillus delbrueckii subsp. bulgaricus (11842, B₁₅Z), Lactobacillus fermentum (DPPMA114, D13), Lactobacillus gasseri (B₃₀W), Lactobacillus helveticus (15009, B₂₆W, PR4), Lactobacillus hilgardii (51B), Lactobacillus paralimentarius (15α, 15β, 16R, 8D, DPPMA238), Lactobacillus paracasei (12H8, 1Hb, B₁₄F₅, B₁₈S, B₂₅F₃, PF6, B₆₁F₆), Lactobacillus pacarbuckneri (B₁₀F₅, B₄₈F₃, B₄₈F₅, B₉F₅ₜ, BF₁, BF₂), Lactobacillus paraplantarum (4DE, DPPMA667), Lactobacillus pentosus (BCF, 12H5, 12H6), Lactobacillus plantarum (14H4, 17N, 19A, 1A7, 2A, 30, 3DM, 4H1, 4H10, DB200, DPPMASL33, DPPMA24W), Lactobacillus rhamnosus (11, 19, DPPMAAZ1, DPPMAAZ21), Lactobacillus sakei (91, SAL1, SAL18), Lactobacillus rossiae (10A, 15R, 3D, 5C1, 5α, CF51, CI35, CR20, E18), Lactobacillus sanfranciscensis (16α, A17, BB12, DE9, E19, H10), Lactococcus lactis subsp. lactis (10γ), Pediococcus pentosaceus (C₁₆F₅, C₂₅F₃, C₃₀F₅ₜ, C₆F₅, C₇F₃, C₉F₅ₜ, C₂₉F₅) and Weissella cibaria (10XA16, 3XA4, 5S, 5XF12) species have been used in the present study. All the biotypes belong to the Collezione di Colture del Dipartimento di Protezione delle Piante e Microbiologia Applicata dell'Università degli Studi di Bari, and have been previously isolated from food matrices (natural yeast for bread-making and cheeses). Biotypes of lactic acid bacteria have been propagated at 30° C. for 24 h in MRS medium (Oxoid, Basingstoke, United Kingdom) at 30 or 37° C. for 24 h.

Cells cultured for 24 h, collected by centrifugation (10,000×g for 15 min at 4° C.), washed two times with phosphate buffer 50 mM, pH 7.0 and re-suspended in water at cell density of log 9.0 ufc/ml have been used for β-glucosidase activity assay. β-glucosidase activity has been quantified as p-nitrophenol released from p-nitrophenol-β-D-glucopyranoside (pNPG) substrate (Sigma Aldrich Chemical Corporate, St. Louis, Mo., USA). 900 μl of pNPG (final concentration) in phosphate buffer 0.5 M, pH 7.5, and 100 μl of cell suspension have been used for assay. The mixture has been incubated at 40° C. and the reaction blocked by heat treatment at 95° C. for 5 min. Absorbance has been measured at 410 nm. One β-glucosidase unit (U) activity has been defined as the enzyme amount needed in order 1 nmol/min of p-nitrophenol to be released under assay conditions (De Angelis et al., 2005. Purification and characterization of an intracellular family 3 β-glucosidase from Lactobacillus sanfranciscensis CB1. Ital. J. Food Sci. 17:131-142).).

### EXAMPLE 2

### Preparation and Fermentation of Soya Milk

Biological Soya (organic farming soybean, OFS) (ECorNaturaSi, Verona, Italy), soy protein isolate (SPI) (Supro Soja 80 Aptonia, Villeneuve d' Ascq, France) and various commercial preparations of soya flours (Cargill Texturizing Solutions Soy Protein, Gent, Belgium) have been used for production of soya milk. OFS has been washed and left standing over night in distilled water. Wet and swollen soya has been manually decorticated, diluted with warm water (about 90° C.), at 1:10 ratio, and homogenised with PBI International homogeniser (Milan, Italy). The homogenization has been carried out at 10,000×g for 2 min, followed by 1 min pause and again treated at 14.000×g for 2 min. The suspension has been centrifuged (7,000×g, 10 min at 4° C.) and soya milk sterile filtered through 0.22 μm pore size filter (Millipore Corporation, Bedford). The pH was 6.2. SPI has been diluted with distilled water (40° C.), at 0.4:10 ratio, and thermally treated at about 55° C. for 30 min under stirring (120 rpm). After cooling at room temp., the pH was adjusted at 6.7 using NaOH 5 M (Tsangalis et al. 2002). Sterilization has been carried out in autoclave at 121° C. for 15 min. The commercial soya flour preparations have been diluted with distilled water (40° C.), at 1:10 ratio, according to method described by Chun et al. (Chun et al., 2007. Conversion of isoflavone glucoside to aglycones in soymilk by fermentation with lactic acid bacteria. J. Food Sci. 72:39-44). pH value was about. 6.3. Sterilization has been carried out in autoclave at 121° C. for 15 min.

Different soya milk types have been inoculated (1-4%, v/v) with a mixed cell suspension of 4 lactic acid bacteria selected on the basis of β-glucosidase activity. Initial cell density of each 4 lactic acid bacteria biotypes was log 7.0 ufc/ml. Fermentation has been carried out at 30° C. for 96 h under stirring (120 rpm). For human intestinal cell assay, soya milk has been frozen-dried, re-suspended in DMEM culture medium and sterile filtered.

### EXAMPLE 3

### Monitoring of the Lactic Acid Bacteria, Determination of Isoflavones-Aglicones, Equol and Lunasin

The monitoring of lactic acid bacteria used as mixed starter (Lactobacillus plantarum DSM 23755 corresponding to DPPMASL33, Lactobacillus plantarum DSM 23756 corresponding to DPPMA24W, Lactobacillus fermentum DSM 23757 corresponding to DPPMA114 and Lactobacillus rhamnosus DSM 23758 corresponding to DPPMAAZ1) during the fermentation of the various soya milk types has been carried out using RAPD-PCR technique. Two primers (Invitrogen, Milan, Italy), with arbitrarily selected sequences (P7 5′ AGCAGCGTGG 3′(SEQ ID No:1), and M13, 5′ GAGGGTGGCGGTTCT 3′ (SEQ ID NO:2)), randomly amplifying different regions of plasmid and chromosomal bacterial DNA (De Angelis et al., 2001. Characterization of non-starter lactic acid bacteria (NSLAB) from ewes' Italian cheeses based on phenotypic, genotypic and cell-wall protein analyses. Appl. Environ. Microbiol. 67:2011-2020; Rossetti e Giraffa, 2005. Rapid identification of dairy lactic acid bacteria by M13-generated, RAPD-PCR fingerprint databases. J. Microbiol. Met. 63:135-144) have been used for typizing.

The extraction of isoflavones-aglicones and equol from fermented soya milk samples has been carried out according to method described by Otieno and Shah (Otieno and Shah, 2007. A comparison of changes in the transformation of isoflavones in soymilk using varying concentrations of exogenous and prebiotic-derived endogenous β-glucosidases. J. Appl. Microbiol. 103:601-612). HPLC chromatographic analysis for the determination of compounds has been carried out according to method described by Maubach et al. (Maubach et al., 2003.

Quantitation of soy-derived phytoestrogens in human breast tissue and biological fluids by high-performance liquid chromatography. J. Chromatogr. 784:137-144).

The determination of lunasin in soya milk obtained from biological soya flour before and during the fermentation has been carried out by HPLC chromatography using an AKTA Purifier system (GE Healthcare) equipped with C18 Xterra Waters column and 214 nm UV detector, eluting with mixture solvent consisting of 5% ACN+0.05% TFA (eluent A) and ACN+0.05% TFA (eluent B) (Wang et al. 2008. Analysis of soybean protein derived peptides and the effect of cultivar, environmental conditions, and processing of lunasin concentration in soybean and soy products. J. AOAC Intern. 91:936-944). Synthetic lunasin has been used as standard (EZBiolab Inc., Carmel, 1N, USA).

### EXAMPLE 4

### Tests on Reconstituted Epidermis and TEER Measurement (Transepithelial Electric Resistance)

Human reconstituted epidermis SkinEthic® (Reconstructed Human Epidermis) consists of normal keratinocytes of human epidermis as a multilayer. It is completely differentiated epidermis after culture of human keratinocytes in a chemically defined medium (MCDM 153), without bovine foetal serum addition, on inert porous polycarbonate support at air-liquid interface for 17 days. At this growth stage the morphologic analysis shows a vital multi-stratified epidermis and a corneous layer consisting of more than ten compact cellular layers. Human reconstituted epidermis SkinEthic® has been used according to previously described procedures (Di Cagno et al., 2009. Synthesis of γ-amino butyric acid (GABA) by Lactobacillus plantarum DSMZ19463: functional grape must beverage and dermatological application. Appl Biotechnol Microbiol DOI: 10.1007/s00253-009-23704).

TEER measurement has been executed using Millicell-ERS Volthommeter (Millipore, Billerica, Mass.). Measurement has been expressed in Ohms×cm².

### EXAMPLE 5

### Tests on Caco-2/TC7 Cells

Human origin Caco-2 cells (clone TC7) have been cultured in Dulbecco (DMEM) medium, added with bovine foetal serum (10%), not essential amino acids (1%), gentamycin/streptomycin (50 μg/ml), glutamine (2 mM) and 4-2-hydroxyethyl-1-piperazinyl-ethanesulfonic acid (1%) (Di Cagno et al., 2010. Quorum sensing in sourdough Lactobacillus plantarum DC400: induction of plantaricin A (PlnA) under co-cultivation with other lactic acid bacteria and effect of PlnA on bacterial and Caco-2 cells. Proteomics in press). The cells viability has been determined by uptake assay of Neutral Red dye (Balls et al., 1987. Approaches to validation alternative methods in toxicology. In: Goldber A. M. (Ed). N.Y. Academic Press pp. 45-58). After treatment for 24-72 h with the different preparations, the cells have been washed with PBS buffer and incubated for 4 h at 37° C. with Neutral Red solution (33 mg/l). Then the cells have been washed again with PBS buffer and treated with lysis solution (50% ethanol in water containing 1% acetic acid). Plate reading has been carried out using Novapath reader (Biorad, Hercules, Calif.) (Di Cagno et al., 2010).

The nitric oxide release (NO) from Caco-2/TC7 cells has been determined by measuring the oxidation products, i.e. nitrite and nitrate. After incubation with the different preparations, the supernatant of cell cultures has been mixed with an equal volume of Griess reagent (1%, p/v, sulfanilic acid in 0.5 M HCl and 0.1%, p/v, N-1-naphthylethylendiamine hydrochloride). After 30 min of incubation at room temp., the absorbance at 540 nm has been measured Nitrite concentration has been determined with reference to standard curve prepared with sodium nitrite.

For TEER measurement Caco-2/TC7 cells have been inoculated (7.5×10⁴ cell/ml) in a microplate container with 24 cells and a polyethylene filter (0.4 μm pore size). Before the treatment, the cells have been incubated for 21 days at 37° C. The treatments with various preparations have been carried out for 18, 24 and 48 h. Integrity of cellular layer then has been determined by means of TEER measurements.

For measurement of released interleukin-8 (IL-8) Caco-2/TC7 cells have previously been incubated (24 h) with interleukin-1β and then stimulated for further 24 h with the different preparations. The synthesis of pro-inflammatory IL-8 has been determined by ELISA assay (Bender MedSystems). The quantification has been carried out using a standard curve according to .kit instructions

Results

(1) Selection of Lactic Acid Bacteria on the Base of β-Glucosidase Activity

Preliminarily, β-glucosidase activity of 103 biotypes of lactic acid bacteria isolated from food matrices has been tested on pNPG synthetic substrate. The activity changed from 0 to 202.3 U (FIG. 1). Forty-eight biotypes belonging mostly to L. alimentarius, L. brevis, L. casei, L. delbrueckii subsp. bulgaricus, L. helveticus, L. hilgardiii, L. paralimentarius, L. paraplantarum, L. pentosus, L. sanfranciscensis, Lc. lactis subsp. lactis, L. parabuchneri e W. cibaria species did not displayed β-glucosidase activity. The activity average value was 3 U, and values corresponding to 25° and 75° data percentile were 0 and 45.5 U. Twenty-five biotypes belonging to different species of lactic acid bacteria have displayed β-glucosidase activity higher than 55 U. L. plantarum DPPMA24W, L. fermentum DPPMA114, L. rhamnosus DPPMAAZ1 and L. plantarum DPPMASL33 have displayed higher activities (202.35±7.08, 163.15±6.52, 146.60±5.84 and 144.34±7.19 U, respectively). The values of β-glucosidase activity for these biotypes have been out of box plot error bar. Based on these result the four lactic acid bacteria have been selected and used for the formulation of a mixed starter to be used for fermentation of the various soya milk types.

(2) Fermentation of Soya Milk and Synthesis of Functional Compounds

The chemical composition, protein dispersibility index and particle size of various soya flour types used for the preparation of soya milk are reported in Table 1. Table 1 shows the chemical composition, protein dispersibility and particle size of 14 soya flours used for functional compound production by selected mixed starter comprising Lactobacillus plantarum DSM 23755 corresponding to DPPMASL33, Lactobacillus plantarum DSM 23756 corresponding to DPPMA24W, Lactobacillus fermentum DSM 23757 corresponding to DPPMA114 and Lactobacillus rhamnosus DSM 23758 corresponding to DPPMAAZ1.

TABLE 1
Chemical composition, protein dispersibility index and granulometry of commercial soya flours

| | | | | Protein | | |
|---|---|---|---|---|---|---|
| | Proteins | Lipids | Fibers | dispersibility | Granulometry | |
| Soya Flour | (%) | (%) | (%) | index | (mesh)* | Description |
| OFS** | 13.1 | 6.7 | 1.1 | 65.3 | 160 | Manually decorticated |
| | | | | | | seeds |
| SPI*** | 83 | 4.4 | 1.5 | 20.1 | 72 | Protein isolated from soya, |
| | | | | | | aromatized with vanilla |
| | | | | | | extract, aspartame |
| | | | | | | edulcorated |
| Prolia 68237 | 54 | 0.95 | 3.5 | 70 | 200 | De-fatted, mild heat |
| | | | | | | treatment |
| Prolia 68238 | 55 | 1.1 | 3.5 | 77.5 | 200 | De-fatted, not toasted |
| Provasoy 68288 | 54 | 1.25 | 3.5 | 22.5 | 100 | De-fatted, enzymatically |
| | | | | | | bitterness made |
| Provasoy 68290 | 54 | 1.25 | 3.5 | 22.5 | 200 | De-fatted, enzymatically |
| | | | | | | bitterness made |
| Provasoy 68282 | 54.2 | 1.0 | 3.5 | 22.5 | 100 | De-fatted, enzymatically |
| | | | | | | bitterness made |
| Provafull 8147 | 39.0 | 21.0 | 3.5 | 10.3 | 72 | Toasted |
| Soy flour | 40 | 2 | 20 | 21.6 | 120 | Toasted |
| Soy semolina | 38 | 22 | 12.4 | 18.6 | 120 | Decorticated and toasted |
| Soy gritz | 38 | 22 | 20 | 16.1 | 11 | Decorticated and toasted |
| Full-fat soy flour | 38.2 | 23 | 16.7 | 19.3 | 100 | Mechanically decorticated |
| Low-fat soy flour | 45.6 | 11.7 | 18.2 | 20.1 | 100 | Extruded, mechanically |
| | | | | | | milled |

*Mesh, mesh/pound;
**OFS, organic farming soy;
***SPI, soy protein isolate

Fourteen soya milk types have been fermented using selected mixed starter comprising L. plantarum DPPMA24W and DPPMASL33, L. fermentum DPPMA114 and L. rhamnosus DPPMAAZ1. All the substrates have been subjected to a process of lactic acidification (FIG. 2A). After 96 h of fermentation, ΔpH values changed from 0.59±0.06 to 1.19±0.09, for soya milk types obtained from Provasoy 68288 and Low-fat soy flours, respectively. ΔpH average value was 0.93, and the range corresponding to 25° and 75° data percentile value was 0.79 and 1.01. After fermentation, pH values for all soya milk types were within 5.1-5.3 range.

Lactic acid bacteria grew during the fermentation of all soya milk types (FIG. 2B). Δlog ufc/ml values changed from 0.99±0.29 to 1.61±0.30, for soya milk types from Full-fat and Low-fat soy flours, respectively. Average value of cell density increase was 1.31 Δlog ufc/ml, corresponding to cell density absolute value of log 8.31 ufc/ml. Range corresponding to 25° and 75° percentile data value was 1.21 and 1.43. Growth of lactic acid bacteria was complete over 24-36 h of incubation. As determined by RAPD-PCR typizing, all four biotypes of lactic acid bacteria used in mixed starter grew on various soya milk types up a similar cell density.

Initial concentration of conjugated isoflavones in various soya milk types changed from 142.3±12.5 to 171.5±10.8, 102.2±8.6 to 123.0±11.3, and from 10.5±1.1 to 18.0±0.9 mM, respectively for daidzin, genistin and glycitin. On the contrary, low concentrations (from 0 to 7.8±0.5 μM) of aglicones have been observed in various soya milk types (Table 2). Table 2 shows the concentration (μM) of isoflavones-aglicones (daidzein, genistein and glycitein) and equol during the fermentation of soya milks, obtained from 14 various flour types, using selected mixed starter. Data are the average of three triplicate experiments.

TABLE 2
Concentration (μM) of isoflavones-aglicones and equol synthesized on various soya milk
types during 96 h of fermentation.

| | Daidzein | | | | |
|---|---|---|---|---|---|
| Soya | Time (h) | | | | |
| milk | 0 | 24 | 48 | 72 | 96 |

Organic farming soy

| | | | | | |
|---|---|---|---|---|---|
| OFS | 6.1 ± 0.7e | 40.9 ± 4.8h | 48.7 ± 3.5k | 53.8 ± 5.3i | 57.0 ± 4.0m |

Soy protein isolate

| | | | | | |
|---|---|---|---|---|---|
| SPI | 4.9 ± 1.1c | 23.2 ± 2.3g | 31.1 ± 3.5j | 35.4 ± 2.6g | 36.6 ± 2.2j |

Commercial soya flours

| | | | | | |
|---|---|---|---|---|---|
| Prolia | 4.7 ± 0.3c | 8.70 ± 0.6f | 26.3 ± 1.0h | 40.1 ± 2.4h | 46.4 ± 1.7k |
| 68237 | | | | | |
| Prolia | 5.9 ± 0.8e | 9.8 ± 1.7 | 29.5 ± 2.0i | 43.3 ± 2.7h | 50.7 ± 2.1l |
| 68238 | | | | | |
| Provasoy | 3.1 ± 0.6b | 6.3 ± 0.4d | 14.9 ± 0.7d | 19.3 ± 1.8e | 21.6 ± 1.2g |
| 68288 | | | | | |
| Provasoy | — | 3.5 ± 0.3b | 7.5 ± 0.6b | 11.0 ± 1.1c | 12.6 ± 0.7c |
| 68290 | | | | | |
| Provasoy | 2.0 ± 0.5a | 4.3 ± 0.8c | 7.9 ± 1.3b | 10.2 ± 0.7b | 11.4 ± 1.0b |
| 68282 | | | | | |
| Provasoy | 3.1 ± 0.2b | 7.1 ± 0.6e | 12.6 ± 1.0c | 15.7 ± 0.5d | 18.5 ± 0.8e |
| 68280 | | | | | |
| Provafull | — | 9.0 ± 0.6f | 15.7 ± 0.9e | 22.8 ± 1.2e | 26.3 ± 0.5h |
| 68147 | | | | | |
| Soy flour | — | 0.8 ± 0.4a | 7.1 ± 0.8b | 11.0 ± 0.7c | 13.4 ± 0.5d |
| Soy | — | 7.5 ± 0.3e | 17.3 ± 1.2f | 23.2 ± 1.4e | 26.3 ± 0.9h |
| semolina | | | | | |
| Soy gritz | — | 4.7 ± 0.3c | 17.7 ± 0.5f | 25.6 ± 1.3f | 28.3 ± 1.0i |
| Full-fat soy | 2.7 ± 0.6b | 3.5 ± 0.5b | 3.9 ± 0.2a | 3.9 ± 0.5a | 3.9 ± 0.8a |
| flour | | | | | |
| Low-fat soy | 5.1 ± 0.9d | 11.0 ± 0.4g | 18.5 ± 1.0g | 19.7 ± 1.6 | 20.1 ± 1.2f |
| flour | | | | | |

| | Genistein | | | | |
|---|---|---|---|---|---|
| Soya | Time (h) | | | | |
| milk | 0 | 24 | 48 | 72 | 96 |

Organic farming soy

| | | | | | |
|---|---|---|---|---|---|
| OFS | 3.6 ± 1.4b | 98.8 ± 8.9i | 125.4 ± 12.1j | 136.9 ± 10.3l | 140.3 ± 9.4k |

Soy protein isolate

| | | | | | |
|---|---|---|---|---|---|
| SPI | 7.4 ± 1.8c | 35.2 ± 2.1h | 64.0 ± 3.6i | 75.1 ± 3.1i | 75.9 ± 2.9h |

Commercial soya flours

| | | | | | |
|---|---|---|---|---|---|
| Prolia | 7.8 ± 0.5d | 21.1 ± 1.3f | 62.2 ± 3.2i | 88.1 ± 6.5j | 94.0 ± 5.3i |
| 68237 | | | | | |
| Prolia | 7.0 ± 0.6c | 24.4 ± 2.0g | 58.2 ± 3.7h | 93.6 ± 6.3k | 102.9 ± 6.4j |
| 68238 | | | | | |
| Provasoy | — | 9.6 ± 0.4d | 25.9 ± 1.4f | 35.1 ± 0.7h | 38.1 ± 0.8g |
| 68288 | | | | | |
| Provasoy | — | 4.8 ± 0.5a | 16.3 ± 0.5e | 22.6 ± 0.8f | 24.8 ± 1.0e |
| 68290 | | | | | |
| Provasoy | — | 4.8 ± 0.3a | 7.4 ± 0.5b | 11.5 ± 0.3c | 14.4 ± 0.6c |
| 68282 | | | | | |
| Provasoy | 2.2 ± 0.1a | 6.3 ± 0.6c | 9.6 ± 0.7d | 17.0 ± 0.4e | 18.9 ± 0.7e |
| 68280 | | | | | |
| Provafull | — | 6.7 ± 0.3c | 10.0 ± 0.6d | 14.4 ± 0.2d | 16.6 ± 0.4d |
| 68147 | | | | | |
| Soy flour | — | 4.1 ± 0.3a | 10.0 ± 0.4d | 11.5 ± 0.2c | 11.8 ± 0.5b |
| Soy | — | 5.9 ± 0.2b | 9.2 ± 0.5c | 10.7 ± 0.6b | 11.1 ± 0.8b |
| semolina | | | | | |
| Soy gritz | 7.0 ± 0.3c | 16.3 ± 0.8e | 28.5 ± 2.1g | 30.3 ± 3.2g | 32.9 ± 1.7f |
| Full-fat soy | — | — | — | — | — |
| flour | | | | | |
| Low-fat soy | — | 4.4 ± 0.3a | 6.3 ± 0.4a | 7.4 ± 0.6a | 7.8 ± 0.4a |
| flour | | | | | |

| | Glycitein | | | | |
|---|---|---|---|---|---|
| Soya | Time (h) | | | | |
| milk | 0 | 24 | 48 | 72 | 96 |

Organic farming soy

| | | | | | |
|---|---|---|---|---|---|
| OFS | 11.3 ± 0.5g | 3.4 ± 1.2b | 17.2 ± 0.9i | 19.3 ± 1.6h | 20.4 ± 1.0h |

Soy protein isolate

| | | | | | |
|---|---|---|---|---|---|
| SPI | — | 2.3 ± 0.7a | 19.7 ± 1.3j | 19.8 ± 0.6h | 20.1 ± 0.8h |

Commercial soya flours

| | | | | | |
|---|---|---|---|---|---|
| Prolia | 6.7 ± 1.0d | 5.9 ± 1.5d | 17.6 ± 2.1i | 21.5 ± 3.2i | 23.9 ± 2.4i |
| 68237 | | | | | |
| Prolia | 6.3 ± 0.4d | 5.6 ± 1.2d | 14.8 ± 3.2h | 21.1 ± 1.8i | 22.5 ± 1.3i |
| 68238 | | | | | |
| Provasoy | — | 4.2 ± 0.5c | 7.0 ± 0.8c | 9.8 ± 0.6b | 10.5 ± 0.4c |
| 68288 | | | | | |
| Provasoy | — | 5.6 ± 0.5d | 8.1 ± 0.7d | 9.8 ± 0.6b | 10.2 ± 0.5c |
| 68290 | | | | | |
| Provasoy | 7.4 ± 0.5f | 9.8 ± 0.7f | 12.3 ± 0.6f | 14.4 ± 1.3e | 15.5 ± 1.1f |
| 68282 | | | | | |
| Provasoy | 6.0 ± 0.5d | 8.8 ± 0.4e | 13.7 ± 0.4h | 16.9 ± 1.1g | 17.6 ± 0.8g |
| 68280 | | | | | |
| Provafull | — | 3.2 ± 0.2b | 4.9 ± 0.5a | 6.0 ± 0.7a | 6.7 ± 0.5a |
| 68147 | | | | | |
| Soy flour | 3.9 ± 0.2b | 12.0 ± 0.7h | 14.1 ± 1.0h | 15.1 ± 0.8f | 17.2 ± 0.6g |
| Soy | — | 3.5 ± 0.1b | 5.6 ± 0.3b | 6.3 ± 0.5a | 7.4 ± 0.2b |
| semolina | | | | | |
| Soy gritz | 3.2 ± 0.3a | 9.8 ± 0.2f | 10.9 ± 0.7e | 11.6 ± 0.6c | 11.6 ± 0.3d |
| Full-fat soy | 6.7 ± 0.1e | 10.2 ± 0.6g | 12.0 ± 0.5f | 12.7 ± 0.4d | 13.7 ± 0.9e |
| flour | | | | | |
| Low-fat soy | 4.6 ± 0.2c | 9.5 ± 0.4f | 13.0 ± 0.9g | 14.1 ± 0.6e | 14.8 ± 0.5f |
| flour | | | | | |

| | Equol | | | | |
|---|---|---|---|---|---|
| Soya | Time (h) | | | | |
| milk | 0 | 24 | 48 | 72 | 96 |
| Organic farming soy | | | | | |
| OFS | — | 7.4 ± 1.1f | 12.3 ± 1.8g | 22.3 ± 1.3f | 37.3 ± 1.5g |
| Soy protein isolate | | | | | |
| SPI | — | — | — | — | — |
| Commercial soya flours | | | | | |
| Prolia | — | 6.1 ± 0.8e | 9.3 ± 1.2e | 11.2 ± 1.4d | 18.5 ± 0.9e |
| 68237 | | | | | |
| Prolia | — | 5.5 ± 1.3d | 10.9 ± 1.0f | 18.7 ± 1.4e | 20.0 ± 1.1f |
| 68238 | | | | | |
| Provasoy | — | 3.2 ± 0.2b | 5.3 ± 0.6b | 10.4 ± 0.5d | 13.6 ± 0.8c |
| 68288 | | | | | |
| Provasoy | — | — | — | — | — |
| 68290 | | | | | |
| Provasoy | — | — | — | — | — |
| 68282 | | | | | |
| Provasoy | — | 4.4 ± 0.5c | 7.4 ± 0.1d | 10.6 ± 0.7d | 14.5 ± 0.3d |
| 68280 | | | | | |
| Provafull | — | 3.4 ± 0.3b | 5.6 ± 0.4b | 8.5 ± 0.2b | 10.3 ± 0.5b |
| 68147 | | | | | |
| Soy flour | — | — | — | — | — |
| Soy | — | 3.1 ± 0.9b | 6.3 ± 0.6c | 9.2 ± 0.7c | 10.3 ± 0.2b |
| semolina | | | | | |
| Soy gritz | — | 0.4 ± 0.4a | 3.5 ± 0.2a | 5.5 ± 0.3a | 7.6 ± 0.1a |
| Full-fat soy | — | — | — | — | — |
| flour | | | | | |
| Low-fat soy | — | — | — | — | — |
| flour | | | | | |

With the exception of soya milk obtained from Full-fat soy flour, the concentration of aglicones increased during the incubation for all soya milk types. After 96 h of incubation, the highest concentration of daidzein has been observed in OFS soya milk (57.0±4.0 μM, corresponding to 1.45 mg/100 ml), followed by Prolia 68238 (50.7±2.1 μM) and Prolia 68237 (46.4±1.7 μM). Also final highest concentration of genistein was in above said three soya milk types (140.3±9.4-3.9 mg/100 ml, 102.9±6.4 and 94.0±5.3 μM, for OFS, Prolia 68238 and 68237, respectively). Compared to others aglicones, the glycitein concentration was lower in all soya milk types. Highest concentration of glycitein was in Prolia 68237 and 68238, and OFS soya milk types (23.9±2.4, 22.5±1.3 and 20.4±1.0 μM-0.58 mg/100 ml, respectively). In the case of soya milk produced from biological soya flour (OFS) the conversion rate of conjugated isoflavones to corresponding aglicones was 0.72, 0.85 and 0.98, for daidzin to daidzein, genistin to genistein and glycitin to glycitein. Although the concentration of aglicones increased during the incubation, after 24 h hydrolysis rates of all three conjugated isoflavones were in 1.0-0.95 range.

Before the incubation, the presence of equol has not been detected in no type of soya milk (Table 2). Various soya milk types were unable to synthesise equol during fermentation process. After 96 h of incubation, the highest concentration of equol has been determined in Prolia 68238 and 68237 soya milk types (20.0±1.1 and 18.5±0.9 μM, respectively) and above all in OFS soya milk (37.3±1.5 μM, corresponding to 0.9 mg/100 ml). Due to the simultaneous synthesis of daidzein, it was not possible to determine the conversion rate thereof to equol. Various studies have considered the use of potentially probiotic bacteria, isolated from human fecal material, in order to enrich soya milk with isoflavones-aglicones (Chun et al., 2007. Conversion of isoflavone glucoside to aglycones in soymilk by fermentation with lactic acid bacteria. J. Food Sci. 72:39-44; Donkor and Shah 2008. Production of β-glucosidase and hydrolysis of isoflavone phytoestrogens by Lactobacillus acidophilus, Bifidobacterium lactis and Lactobacillus casei in soymilk. J. Food Sci. 73:15-20; Pham and Shah 2007. Biotransformation of isoflavone glycosides by Bifidobacterium animalis in soymilk supplemented with skim milk powder. J. Food Sci. 72:316-324; Tsangalis et al., 2002; Tsangalis et al., 2004; Wei et al., 2007. Using Lactobacillus and Bifidobacterium to product the isoflavone algycones in fermented soymilk. Int. J. Food Microbiol. 117:120-124). Used microorganisms have been exclusively bifidobacteria or various lactic acid bacteria belonging to various species. The present invention has selected four new biotypes corresponding to L. plantarum DPPMA24W and DPPMASL33, L. fermentum DPPMA114 and L. rhamnosus DPPMAAZ1, never used previously for the synthesis of isoflavones-aglicones and equol. Only a limited number of studies has considered also the synthesis of equol during the fermentation of soya milk. Equol has been synthesized in soya milk fermented with bifidobacteria (Tsangalis et al., 2002. Enzymatic transformation of isoflavone phytoestrogens in soymilk by β-glucosidase producing bifidobacteria. Food Res. Int. Sci. 67:3104-3113). After 24 h of fermentation, highest concentration of equol (0.521 mg/100 ml) was synthesized by Bifidobacterium animalis, compared with the production of 0.338 and 0.433 mg/100 ml obtained using Bifidobacterium pseudolongum and Bifidobacterium longum biotypes. OFS soya milk fermented with the mixed starter selected according to this study contained higher concentration of equol, namely 37.3 μM corresponding to 0.9 mg/100 ml.

Based on previously reported results, soya milk produced from biological soya flour (OFS) has been considered the best substrate for the synthesis of isoflavones-aglicones and equol. On the base of our knowledge, no previous study considered the use of soya milk obtained from biologically cultured soya flour for the synthesis of isoflavones-aglicones and equol.

Therefore, the concentration of lunasin using HPLC method (Wang et al. 2008. Analysis of soybean protein derived peptides and the effect of cultivar, environmental conditions, and processing of lunasin concentration in soybean and soy products. J. AOAC Intern. 91:936-944) has been determined. Before the incubation, the lunasin concentration was about. 3.2 mg/100 ml (FIG. 3). During the fermentation, the selected mixed starter favoured a constant increment of lunasin that, at the end of 96 h of incubation, was about 8.4 mg/100 ml. On the base of our knowledge, no previous study considered the concomitant synthesis of isoflavones-aglicones, equol and lunasin in the same preparation consisting of soya milk fermented with lactic acid bacteria. The physiological effects of this bioactive peptide (lunasin) are widely documented in literature (Jeong et al., 2003. Characterization of lunasin isolated from soybean. J Agric Food Chem. 51: 7901-7906; Jeong et al. 2007. The cancer preventive peptide lunasin from wheat inhibits core histone acetylation. Cancer Lett. 255:42-48).

Based on previous results, fermented OFS soy milk was used for assays of cutaneous protection and on intestinal human cells.

(3) Tests on Reconstituted Epidermis and TEER Measurement (Transepithelial Electric Resistance)

OFS soya milk obtained from biological soya flour and fermented with selected mixed starter have been used at equol final concentration of 10 μM for treatment of human reconstituted epidermis according to the SkinEthic® model. This model has been wide experimented and accepted by the scientific community (Di Cagno et al., 2009. Synthesis of γ-amino butyric acid (GABA) by Lactobacillus plantarum DSMZ19463: functional grape must beverage and dermatological application. Appl Biotechnol Microbiol DOI: 10.1007/s00253-009-23704). After treatment for 24 h, TEER measurement has been carried out. This type of analysis, widely accepted by the international scientific community, evaluates the corrosion capacity of tissue taking as a reference the integrity of corneous layer and the barrier function. Particularly, by means of this detection it is possible to obtain information about the presence of a compact lamellar structure at corneous layer level, of integral tight junctions and epidermic thickness. These factors as a whole define an efficient barrier function. FIG. 4 shows as in the presence of fermented OFS soya milk a remarkablel increase (P<0.05) of TEER value is present, demonstrating a protecting action of the molecule at cutaneous level. The same result has been obtained with a mixture of chemically synthesised equol and lunasin.

According to current knowledge this is the first application example of preparation based on soya milk containing isoflavones-aglicones, equol and lunasin demonstrating a stimulation of the cutaneous barrier functions.

(4) Tests on Caco-2/TC7 Cells

With the purpose to test immunomodulating properties of isoflavones-aglicones contained in soya milk produced from biological soya flour (OFS), cytotoxicity against Caco-2/TC7 cells by standard chemical compounds (equol, daidzein, genistein and glycitein) at concentrations of 5-100 μM using Neutral Red (NR) uptake assay, firstly has been evaluated. Genistein, glycitein and equol have shown a behaviour similar to methanol and DMSO (negative control) and did not affect significantly cell proliferation. After 72 h of treatment, daidzein remarkably inhibited (P<0.03) cell proliferation at concentration higher than 100 μM.

Preliminarily, Caco-2/TC7 cells have been treated for 24 h at concentration of 10 μM with the OFS fermented soya milk and diluted at equol final concentration of 10 μM or with not fermented soya milk. These compounds or preparations did not display induction for NO release, showing a behaviour similar to negative control, i.e. methanol and DMSO (FIG. 5). Successively, Caco-2/TC7 cells have been stimulated with INF-γ (1000 U/ml) e LPS (100 ng/ml) per 24 h. This treatment significantly increased (P<0.05) the NO release, thus simulating the inflammatory state of Caco-2/TC7 cells, preventively treated with negative control, daidzein or not fermented OFS soya milk. On the contrary, treatments with equol or fermented OFS soya milk inhibited in a marked manner (P<0.002 and P<0.007, respectively) the NO release. A considerable inhibition of NO release has been also observed using treatments with genistein and glycitein (P<0.05). Since preliminarily it has been demonstrated that concentration (10 μM) of isoflavones-aglicones used in the test is not toxic, the death of Caco-2/TC7 cells has not surely interfered with NO release.

Under culture conditions of this study, Caco-2/TC7 cells develop morphological and functional characteristics of enterocytes, including tight intercellular junctions, integrity thereof being measured by TEER determination. Preliminarily, TEER has been determined in the presence of standard chemical compounds (10-100 μM), fermented OFS soya milk and diluted at equol concentration of 10 μM, or not fermented OFS soya milk. With the exception of equol chemical compound at concentration of 100 μM (1000 U/ml) effects on TEER during 72 h of incubation have not been observed. Treatments of Caco-2/TC7 cells with INF-γ (1000 U/ml) favoured a remarkable decrease (P<0.003) of TEER value (FIG. 6). When Caco-2/TC7 cells stimulated with INF-γ have been treated also with fermented OFS soya milk the negative effect of INF-γ is remarkably attenuated (P<0.007). A negligible effect has been observed in the presence of not fermented OFS soya milk. Genistein, glycitein and above all equol have shown a trend similar to fermented OFS soya milk. Daidzein has not interfered with the negative effect caused by INF-γ.

Interleukin-8 (IL-8) is a member of C—X—C chemokine family and plays a fundamental role in activation of neutrophil cells, thus initiating the inflammatory response. When Caco-2/TC7 cells are subjected to a treatment with inteleukin-1β (2 ng/ml) has been observed a meaningful increment (P<0.001) of IL-8 synthesis (FIG. 7). When Caco-2/TC7 cells, stimulated with interleukin-β, have been subjected also to a treatment with equol and daidzein a meaningful decrement (P<0.005) of IL-8 synthesis has been observed. Highest inhibition of IL-8 synthesis (P<0.001) has been observed by treatment with fermented OFS soya milk. On the contrary, treatments with genistein, glycitein or OFS soya milk fermented did not resulted in (P<0.10) a decrement of IL-8 synthesis.

Reported results clearly show that the anti-inflammatory and stimulating effect to barrier functions of intestinal human cells by fermented OFS soya milk is mainly the result of the presence of equol and some isoflavones-aglicones. An additive effect by lunasin is possible.

(5) Development a Biotechnological Protocol for the Synthesis of Daidzein, Genistein, Glycitein, Equol and Lunasin and Use Thereof in Dermatological Field

As previously outlined in other part of the text, a biotechnological process for the synthesis of isoflavones-aglicones (daidzein, genistein and glycitein), equol and lunasin and use thereof in dermatological field has developed. Said process involves:

a) culture of L. plantarum DPPMA24W and DPPMASL33, L. fermentum DPPMA114 and L. rhamnosus DPPMAAZ1 in pure culture on MRS culture medium;

b) collection, washing and inoculum of the cell suspensions in various soya milk types, preferably, sterile soya milk prepared from soya flour cultured according to agronomic biological methods and laboratory decorticated;

c) fermentation of soya milk by selected mixed starter for 48-96 h, preferably 96 h at 30-37° C., preferably 30° C.;

d) separation of cells by centrifugation. According to a process variant the preparation can also contain lactic acid bacteria cells;

e) dehydration of the preparation by drying or freeze-drying process;

f) preparation of a composition by addition of suitable excipients in order to obtain forms suitable to use by oral or topical administration depending on the cases.

### EXAMPLE 6

### In Vitro Evaluation of Biomass Containing Lunasin Vs Biomass without Lunasin on Stimulation of Hair Growth

In vitro study of biomass containing lunasin (BL) compared to without lunasin (B) as promoter for hair growth.

Material and Methods

Derma papilla cells (DPCs) have been cultured in medium (Dulbecco's modified Eagle's medium, DMEM) containing 2 mM L-glutamine, 1× of antimycotic and antibiotic solution (1000 u g/ml streptomycin sulfate, 1000 unit/ml penicillin G and 2.5 μg/ml amphotericin B) and 10% bovine foetal serum. At confluence the cells have been cultured for 24 hours in DMEM without serum and then treated with various concentrations of biomass containing or not lunasin.

The cell proliferation has been determined by MIT method (Mosmann, 1983). DPCs have been seeded in a 96 well plate (10⁴ cell/well) and incubated for 24 hours adding the substances to be assayed. Absorbance has been measured at 570 nm with an ELISA reader.

Further western blot has been carried out on Bcl2. The proteins have been extracted using buffer containing Tris-HCl 50 mM, pH 7.4, EDTA 2 mM, leuptin 100 μg/ml and 100 NaCl mM.

50 ug of proteins have been loaded and separated by SDS-PAGE. Monoclonal antibodies against Bcl-2, Bax and actin have been diluted 1:500, the antigen-antibody complex has been detected using ECL system and the result analyzed using image densitometry (Bio-Rad GS-700).

Results and Discussions

In the range of tested concentrations (0.01-0.5 μM) lunasin containing biomass induces an increase of in vitro DPCs proliferation according to dose dependent way (p<0.05) (FIG. 1).

The effect of lunasin containing biomass, differently than biomass without lunasin, induces an increase of Bcl-2 protein expression and a decrease of Bax protein expression (FIG. 2).

These data suggest that lunasin containing biomass stimulates the hair growth through proliferative and anti-apoptotic effect thereof on DPCs, could, therefore extend the anagen phase.

1. A process for preparing a fermented soya based mixture, the process comprising
performing soya fermentation using a mixture of Lactobacillus plantarum DSM 23755, Lactobacillus plantarum DSM 23756, Lactobacillus fermentum DSM 23757 and Lactobacillus rhamnosus DSM 23758, to produce a fermented soya based mixture comprising isoflavones-aglicones, equol and lunasin.

2. The process according to claim 1, wherein the performing comprises the following steps:
a) propagating cultures of lactic acid bacteria Lactobacillus plantarum DSM 23755, Lactobacillus plantarum DSM 23756, Lactobacillus fermentum DSM 23757 and Lactobacillus rhamnosus DSM 23758;
b) inoculating soya based substrates with an aqueous suspension of said lactic acid bacteria to provide inoculated cultures;
c) incubating the inoculated cultures at 30-37° C.

3. The process according to claim 2, wherein the inoculating is performed with a aqueous suspension of the lactic acid bacteria in an amount from 1 to 4% of total substrate volume, said aqueous suspension having a cell density of about log 9.0 ufc/ml for every strain.

4. The process according to claim 2, wherein the soya based substrates are selected from the group consisting of soya flour, preferably biological soya flour, soya milk.

5. The process according to claim 2 further comprising step of
d) centrifuging the inoculated culture to remove the lactic acid bacteria cells.

6. The process according to claim 5, wherein the centrifuging is carried out at 10.000×g for 15 min at 4° C.

7. The process according to claim 2 further comprising step of
e) dehydrating the incubated cultures obtained in step c) by drying or freeze-drying.

8. A fermented soya based mixture, comprising isoflavones-aglicones, equol and lunasin, obtainable by the process as defined in claim 1.

9. A pharmaceutical or cosmetic composition comprising the fermented soya based mixture as defined in claim 8 together with one or more pharmaceutical or cosmetically acceptable excipients and/or adjuvants.

10. A food integrator, the food integrator comprising the fermented soya based mixture according to claim 8, as such or in combination with one or more excipients and/or adjuvants.

11. A method to treat skin or intestinal walls of an individual, wherein the method comprises
administering to the individual the fermented soya based mixture according to claim 8 in an effective amount to treat disorders or diseases of skin or intestinal wall.

12. A method to treat skin of an individual, the method comprising
administering to the individual the fermented soya based mixture according to claim 8 in a cosmetically effective amount.

13. A method to treat an individual, the method comprising
administering to the individual the fermented soya based mixture according to claim 8, in an effective amount for treatment of hair loss of the individual.

14. A method to treat an individual, the method comprises
administering to the individual the mixture according to claim 8 in an effective amount to treat alopecia or telogen defluvium.

15. A mixture of lactic acid bacteria Lactobacillus plantarum DSM 23755, Lactobacillus plantarum DSM 23756, Lactobacillus fermentum DSM 23757 and Lactobacillus rhamnosus DSM 23758.

16. Lactobacillus plantarum DSM 23755 lactic acid bacterium.

17. Lactobacillus plantarum DSM 23756 lactic acid bacterium.

18. Lactobacillus fermentum DSM 23757 lactic acid bacterium.

19. Lactobacillus rhamnosus DSM 23758 lactic acid bacterium.

20. The process according to claim 2, wherein the incubating is performed at 30° C., for 48-96 h.

21. The process according to claim 2, wherein the incubating is performed at 30° C. for 96 h.

22. The process according to claim 5 further comprising the step of
e) dehydrating a supernatant obtained by the centrifuging of step d), the dehydrating performed by drying or freeze-drying the supernatant.

23. The pharmaceutical or cosmetic composition of claim 9, wherein the composition is formulated for treatment of disorders or diseases of skin or intestinal wall of an individual.

24. The pharmaceutical or cosmetic composition of claim 9, wherein the composition is formulated for cosmetic use.

25. The pharmaceutical or cosmetic composition of claim 9, wherein the composition is formulated for treatment of hair loss in an individual.

26. The pharmaceutical or cosmetic composition of claim 9, wherein the composition is formulated for treatment of alopecia or telogen defluvium in an individual.
